(19) United States
(12) Patent Application Publication
(10) Pub. No.: US 2022/0400694 A1
(43) Pub. Date: Dec. 22, 2022

(54) BIOPOLYMER EMULSION FOR ACTIVE PACKAGING, USES AND METHOD OF MANUFACTURING

(71) Applicant: Univerzitet U Beogradu, Belgrade (RS)

(72) Inventors: Zorica Brankovic, Belgrade (RS); Jovana Cirkovic, Belgrade (RS); Aleksandar Radojkovic, Belgrade (RS); Goran Brankovic, Belgrade (RS); Jelena Jovanovic, Belgrade (RS); Slobodan Krnjajic, Belgrade (RS); Sonja Veljovic Jovanovic, Belgrade (RS)

(21) Appl. No.: 17/773,585

(22) PCT Filed: Sep. 14, 2018
(86) PCT No.: PCT/RS2018/000013

§ 371 Date: Apr. 29, 2022

(51) Int. Cl. A23B 7/157; A23B 7/154

(52) U.S. Cl. CPC A23B 7/157; A23B 7/154

(57) ABSTRACT

The present invention is in the field of aqueous emulsions that dry into water-insoluble or water-resistant structures that are useful for active packaging, manufactured devices and components, and other applications. The aqueous emulsions of the present invention comprise biopolymers, metal in the form of a salt, nanoparticles or metal oxide nanoparticles, essential oil, and additives such as surfactants and plasticizers. When the components of the emulsion are mixed following the distinctive method of preparation, a water-soluble fluid is obtained, which, upon drying, becomes a water-insoluble or water-resistant solid exhibiting antimicrobial, antioxidative, and other useful properties including tensile strength, elasticity, transparency. The obtained fluid may be applied by spraying, pouring, injecting, 3-D printing, or otherwise formed into a solid product of any geometrical shape including film, foil, or other 3-D shape.

### TECHNICAL FIELD

The present invention is in the field of aqueous emulsions that dry into water-insoluble or water-resistant structures that are useful for active packaging, manufactured devices and components, and other applications. Particularly, the present application discloses the emulsion for products protection, preferably foods or other products vulnerable to any type of oxidation and spoilage, e.g. fresh fruit and vegetables. Moreover, the emulsion may be applied even for protection and packaging of organically grown food or other products that must satisfy extremely strict food safety regulations. More specifically, the present invention relates to emulsions comprising biopolymers, metal in the form of a salt, nanoparticles or metal oxide nanoparticles, essential oil, and additives such as surfactants and plasticizers. When the components of the emulsion are mixed following the distinctive method of preparation, a water-soluble fluid is obtained, which, upon drying, becomes a water-insoluble or water-resistant solid. Said solid product exhibits antimicrobial, antioxidative, and other useful properties including tensile strength, elasticity, transparency. Said solid can be relatively simply and economically manufactured and even more, is safe for human and environmental health. The obtained fluid may be applied by spraying, pouring, injecting, 3-D printing, or otherwise formed into a solid product of any geometrical shape including film, foil, or other 3-D shape. It is known that essential oils exhibit exceptional antimicrobial and antioxidative properties. However, their use is limited due to their short term effect related to their high volatility. Thus, it is one aim of the present invention to reduce the volatility of essential oils and achieve their prolonged effect. This is accomplished, as further explained in detail, by nanoencapsulation of essential oils, as the active components of the emulsion, into biodegradable biopolymer. We have demonstrated that encapsulation provides slow release of essential oil and allows its prolonged effect. The addition of metal to the encapsulating emulsion mixture creates chemical conditions that result in the useful properties of the dried mixture.

### BACKGROUND ART

There are many applications in which it is desirable to create a water-soluble mixture and then to spray, form, inject or otherwise configure that mixture into a water-insoluble or water-resistant solid with various useful properties such as antimicrobial and antioxidative. Such applications include active packaging and 3-D components and devices wherein these properties are advantageous. A discussion of the invention's embodiment in active packaging application is illustrative. Despite the fact that there are numerous investigations in the field of packaging materials for fresh ingredients, it is still a big challenge to make a packaging material using environmentally friendly raw materials, which at the same time are non-toxic to humans, exhibit good mechanical, antimicrobial, antioxidative, fungicide and barrier properties but also are economical to manufacture.

Plastic packaging materials currently commercially available, unfortunately do not inhibit the deleterious microbial growth, allow product oxidation, and are non-degradable.

CN 106750580A describes antibacterial and mechanical properties of the food packaging film comprising chitosan, gelatin, cinnamon oil and glycerin. Moreover, CN 106750580A provides an antibacterial edible food packaging film, which is environmentally friendly and exhibits improved antimicrobial and antibacterial properties. However, the problem of high volatility of essential oils has not been addressed.

CN 107163349A discloses a three layer composite film comprising chitosan, polyphenols, Ginkgobiloba extract, Wisteria extract and sage extract. The invention aims to provide a plastic wrap exhibiting antibacterial and antioxidative properties, which can significantly prolong cold storage of food, especially fresh food.

DE 19532489 A1 suggests the use of Si, Ti and Al oxides and various synthetic polymers for providing antimicrobial packaging material as well as their use in the process of producing an antimicrobial packaging material employing various antibiotic compounds that may be adsorbed or embedded in binder form.

EP 2025620 A1 relates to an active packaging that inhibits food pathogens either by means of the generation of an active atmosphere or by means of direct contact. The active packaging comprises a support made from paper, cardboard, cork, aluminum or wood and an active coating thereof. The coating consists of a formulation of paraffin and natural plant extracts, where paraffin is used as an anti-humidity barrier but also as a carrier of pathogen inhibitor agents, and where cinnamon essential oil is incorporated into the paraffin. The disclosure mentions that essential oils are liquids that contain relative volatile compounds, and suggests the use of surfactants or agents that fixate the volatile compounds in order to solve this problem. Moreover, the study of anti-microbial activity over time is disclosed, where the total inhibition was observed with C. albicans and A. flavus up to 71 days. However, Gram-negative bacteria were not studied, since the previous results show that the inhibition is only partial.

Hence, the disadvantage of the prior art is that still it does not provide a material for active packaging and products protection comprising essential oils, that would exhibit prolonged and broad antimicrobial activity, antioxidative, and other essential properties, but also demonstrates sufficient tensile strength, elasticity, transparency, allows simple and cost-effective manufacture, and above all, is safe for human and environmental health.

### DISCLOSURE OF INVENTION

Therefore, the object of the present invention is to provide a material that is water-soluble when initially mixed, water-insoluble or water-resistant upon drying after spraying, forming, or configuring, and which utilizes the mechanical, antimicrobial, antioxidative, and other useful properties for various applications including 3-D apparatus and components, as well as packaging for product protection, particularly foods, that comprises essential oils exhibiting excellent antimicrobial and antioxidative properties, with reduced volatility and thereby their prolonged effect. Additionally, the material according to the present invention exhibits other essential properties including tensile strength, elasticity, transparency, allows simple and cost-effective manufacture, and above all, is safe for human and environmental health.

The object is solved by the emulsion according to the present invention, which comprises biopolymer, essential oil, a metal (which may be in the form of a salt, nanoparticles or metal oxide nanoparticles), a plasticizer and a surfactant.

The combination of the components provides a number of different and useful properties including encapsulation of essential oils by the biopolymer mixture, which allows their slow release. Described emulsion can be processed into protective coatings by methods of spraying, doctor blade technique, foil casting or it can be 3D printed, extruded, or otherwise molded to any desirable solid. The packaging material as described in the present application can replace existing, commercially available plastic materials that facilitate the development of deleterious microbes, do not inhibit product oxidation, and are non-degradable, which complicates their disposal. The use of the active biodegradable packaging will secure safety of food or any other product vulnerable to any type of oxidation or spoilage. Moreover, it will allow prolonged shelf life of these products. On the other hand, it will resolve environmental issues caused by the use of non-degradable plastic packaging.

### BEST MODES FOR CARRYING OUT OF THE INVENTION

The emulsion of the present invention comprises biopolymer, essential oil, metal (which may be in the form of a salt, nanoparticles or metal oxide nanoparticles), a plasticizer and a surfactant.

Biopolymer is selected from the group comprising: polysaccharides, such as pectin, chitosan, alginate, starches, ligno-cellulosic products (e.g. wood, straws), proteins (such as casein, whey, collagen, gelatin, zein, soya, gluten), lipids (e.g. wax) and combination thereof. Essential oils are introduced in the biopolymer blend as the active components, because they exhibit excellent antimicrobial and antioxidative activity even at low concentrations. The emulsion contains the essential oil selected from the group comprising Allium sativum, Cinnamomum zeylanicum, Cuminum cyminum, Epilobium parviflorum, Lavandula officinalis, Mentha piperita, Ocimum basilicum, Ocimum gratissimum, Origanum majorana, Origanum vulgarae, Pimenta dioica, Pimpinella anisum, Piper betle, Psiadia arguta, Psiadia terebinthina, Rosmarinus officinalis, Salvia desoleana, Salvia sclarea, Satureja, Montana, Thymus vulgaris etc. or their active components: p-cymene, limonene, menthol, eugenol, anethole, estragole, geraniol, thymol, γ-terpinene, cinnamyl alcohol or combination thereof.

In addition, the emulsion comprises metal such as silver, gold, zinc, titanium, calcium, copper, magnesium, which may be in the form of a salt, nanoparticles or metal oxide nanoparticles or combination thereof. In one preferred embodiment, the emulsion contains ZnO nanoparticles or TiO₂ nanoparticles or Zn-acetate or combination thereof, which imbues the resulting dried solid with exceptional antimicrobial, antioxidative, mechanical, and other useful properties.

The emulsion according to the present invention further contains a surfactant selected from the group comprising: polyethoxy-esters, glycerol esters, esters of hexitols and cyclic anhydrohexitols: sorbitan esters (e.g. commercially available SPAN) and their ethoxylated counterparts (e.g. commercially available TWEEN).

In one preferred embodiment of the invention, the biopolymer component of the emulsion is a combination of biopolymers: chitosan and gelatin (C/G) or pectin and gelatin (P/G), where the gelatin content is preferably up to 30 wt %; more preferably from 10 wt % to 20 wt %. The active components of the emulsion including essential oils are present preferably in concentration up to 25 wt %, relative to the biopolymer weight, while metal, which may be in the form of a salt, nanoparticles or metal oxide nanoparticles or combination thereof, may be present in concentration up to 3 wt %, relative to the biopolymer weight.

As additive components, 1% solution of acetic acid for chitosan dissolving may be used. Furthermore, a plasticizer that improves the elasticity of the dried solid, such as glycerol, may be used. Alternatively, plasticizer may be selected from the group comprising sorbitol, xylitol, PEG, PG, sucrose, fatty acids, etc.

In one preferred embodiment, surfactant is Tween 80. In general, surfactant is used for stabilization and emulsion nanoencapsulation. The concentration of the surfactant present must be at least 15 wt %, relative to the weight of essential oil.

Emulsion according to the present invention may be prepared as described in the following examples.

### Example 1

Biopolymer Emulsions Based on Pectin and Gelatin (P/G)

Gelatin is allowed to dissolve in distilled water at 35° C. for 15 min. Pectin is allowed to dissolve in distilled water at 60° C. for 45 min. The two solutions are mixed together in the ratio of pectin/gelatin 80/20 for 10 min. For the purpose of stabilizing the emulsion and improving the elasticity of the films obtained, 50 wt % glycerol (relative to the mass of the dissolved biopolymer) was added to the solution and mixed with the UltraTurrax homogenizer for 10 minutes. Lemon grass essential oil (LG), in concentration up to 25 wt % (relative to the weight of the biopolymer) is added to the biopolymer solution, and the obtained mixture is homogenized for 15 minutes using UltraTurrax. After homogenization, to stabilize the emulsion and ensure the encapsulation of essential oil, 15 wt % Tween 80 (relative to the weight of the essential oil) is added and mixed with UltraTurrax for another 10 minutes. Metal salt, such as Zn-acetate or metal oxide nanoparticles, such as ZnO nanoparticles or TiO₂ nanoparticles, in concentration 1.0 wt % (relative to the mass of the dissolved biopolymer) is added to the obtained emulsion. Finally, the emulsion is homogenized for 30 minutes.

### Example 2

Biopolymer Emulsions Based on Chitosan and Gelatin (C/G):

Chitosan is allowed to dissolve in 1% acetic acid solution at room temperature for 20 h. Gelatine is allowed to dissolve in distilled water at 35° C. for 15 min. The two biopolymer solutions are mixed with chitosan/gelatin (ratio 80/20) and then mixed for 10 min. 50 wt % glycerol (relative to the biopolymer weight) is added to the solution and mixed with the UltraTurrax homogenizer for 10 minutes. Essential oil, e.g. lemon grass, in concentration up to 25 wt % (relative to the biopolymer weight) is added to the biopolymer solution, and the emulsion is homogenized by intensive blending for 15 min using UltraTurrax. After homogenization, in order to stabilize the emulsion and ensure essential oil encapsulation, 15 wt % Tween 80 (relative to the weight of the essential oil) is added and mixed with UltraTurrax for another 10 minutes. Metal salts e.g. Zn-acetate or metal oxide nanoparticles e.g. ZnO nanoparticles, in concentration of 1.0 wt % (relative to the biopolymer weight), are added to the emulsion and finally the emulsion is homogenized for 30 minutes.

Mixing and homogenization procedures could be performed in other suitable ways (ultrasound, mechanical, magnetic, with or without increasing temperature) known to the person skilled in the art, depending on the type of biopolymers and other components.

The emulsion obtained as described above may be applied as a spray. The emulsion is first diluted to achieve the desirable viscosity, and then directly sprayed on the substrate, such as packaging made of paper, cardboard, cork or wood, conventional polymers (plastic), glass or metal (e.g. Al foil) packaging. It may also be applied as a protective layer (i.e. coating or film) on the active packaging for food and other products to be protected or even on any disposable surface, e.g. on the walls of storage rooms or containers. The emulsion as disclosed may be used even in households. Upon spraying, it takes about 30 min to dry the formed coating layer. Moreover, the emulsion prepared as described can be stored in tightly closed dark bottles at room temperature, without direct exposure to day light, for at least 3 months.

In another embodiment, the emulsion is used in the form of a foil, by casting into different molds. The emulsion is cast into a mold and dried at room temperature for up to 24 h depending on the layer thickness and ambient conditions. The foil is then removed from the mold and may be used as a packaging material for wrapping or covering the product to be protected. In another embodiment, the emulsion is used as a protective coating, e.g. film, which is deposited by doctor blade technique with controllable thickness. The doctor blade technique involves casting of the emulsion on a substrate, such as existing packaging, by adjusting the layer thickness via the blade of the instrument. It takes up to couple of hours upon casting for the film to be completely dried, depending on the thickness of the layer and ambient conditions.

In another embodiment, the emulsion is used as a pad with desirable mechanical and slow release properties.

In another embodiment, the emulsion is in form of a 3D printed object.

In another embodiment, the emulsion is injected or poured into a mold of any shape.

In another embodiment, the emulsion is impregnated into existing packaging material.

Tests have been performed that demonstrate the following crucial properties of the solid product obtained from the emulsion of the present invention: self-organizing meta-structure of dried polymeric matrix network, broad antimicrobial activity, antioxidative activity, insecticide activity, moisture-resistance properties, excellent tensile strength, elasticity, transparency, formation of any geometrical shape and structure including films, foils, 3-D objects. Furthermore, the material allows products covered by the dried solid to be protected from microbes or oxidation either by fumigant effect or direct contact.

Moreover, the emulsion is composed of environmentally rational, biodegradable, renewable, natural ingredients, which are also economical. In addition, the emulsions of the invention can be stored, unchanged at room temperature for at least 3 months.

### ABTS Test—Antioxidative Properties of the Emulsion

### 2,2′-Azino-bis(3-ethylbenzothiazoline-6-sulfonic acid) (ABTS)

Total antioxidative activity of the emulsion according to the present invention is determined by using the ABTS test. The reaction mixture contains 2 mM ABTS, 15 mM H₂O₂ and 0,625 mM horseradish peroxidase in 50 mM phosphate buffer (pH 7.5) at room temperature. 10-fold dilution in water is prepared and the antioxidative activity is presented related to the activity of L-ascorbic acid as a standard and is expressed as an equivalent. The assessment of the antioxidative capacity of the emulsions for capture ABTS.+radicals is measured at 730 nm. Table 1 demonstrates the antioxidative properties of the emulsion based on pectin and gelatin or chitosan and gelatin and with addition of different percentage of essential oil (e.g. LG), metal oxide nanoparticles (e.g. ZnO, TiO₂) or salts (e.g. Zn-acetate).

TABLE 1

| | eqAsc | Standard |
|---|---|---|
| Emulsion | (mM) | error |
| Pectin + 12.5 wt % LG | 0.73 | 0.05 |
| Pectin + 25 wt % LG | 0.84 | 0.00 |
| P/G + 12.5 wt % LG | 0.59 | 0.00 |
| P/G + 25 wt % LG | 0.87 | 0.04 |
| P/G + 1 wt % ZnO | 0.66 | 0.00 |
| P/G + 1 wt % Zn-acetate | 0.66 | 0.03 |
| P/G + 1 wt % TiO₂ | 0.68 | 0.01 |
| P/G + 25 wt % LG + 1 wt % ZnO | 0.89 | 0.02 |
| P/G + 25 wt % LG + 1 wt % Zn-acetate | 0.99 | 0.02 |
| P/G + 25 wt % LG + 1 wt % TiO₂ | 0.95 | 0.00 |
| Chitosan + 25 wt % LG | 0.29 | 0.01 |
| C/G | 0.31 | 0.03 |
| C/G + 25 wt % LG | 0.46 | 0.03 |
| C/G + 1 wt % ZnO | 0.35 | 0.00 |
| C/G + 25 wt % LG + 1 wt % ZnO | 0.47 | 0.02 |

The data shown in Table 1 indicate that the presence of essential oil (LG) has a positive effect on the antioxidative properties of the emulsions. Additionally, the presence of the metal oxide nanoparticles such as ZnO, TiO₂ does not compromise their antioxidative properties, while the addition of salt (e.g. Zn acetate) even improves the antioxidative capacity of the emulsion. The highest values of the antioxidative activity were reached by simultaneous activity of essential oil and Zn-acetate.

### Antimicrobial Properties of the Emulsion Disk—Diffusion Method of Antimicrobial Activity Testing

Antimicrobial properties were assessed by the disk-diffusion method according to the CLSI (Clinical and Laboratory Standards Institute) M02-A11 (CLSI, 2012) standard. Inoculum is prepared by a suspension of bacterial cultures up to 0.5 McFarland turbidity, which is equivalent to the concentration of 1-2×10⁸ cfu/ml. The Muller-Hinton II agar (CAMHA, Becton, Dickinson and Company, USA) was used as a substrate for all tested bacteria except for Streptococcus agalactiae, for which CAMHA with addition of 5% sheep blood was used. The sterile discs (HI MEDIA, INDIA) with the material according to the present invention applied, were placed on a plate with inoculum.

Table 2 demonstrates antimicrobial properties of the emulsion according to the present invention, based on pectin and gelatin, the emulsion further containing essential oil (LG) and/or ZnO nanoparticles or Zn-acetate. The zone of inhibition is represented in mm and only results above 6 mm were considered as effective, due to disc dimensions. All responses below 6 mm were marked as “-”.

TABLE 2

| | | Streptococcus | | | |
|---|---|---|---|---|---|
| | S. aures | agalactiae | E. coli | P. aeruginosa | P. vulgaris |
| P/G | — | — | — | — | — |
| P/G + 1 wt % ZnO | 8 mm | 12 mm | — | — | — |
| P/G + 1 wt % Zn-acetate | 12 mm | 16 mm | — | — | — |
| P/G + 0.5 wt % Ca-acetate | 12 mm | 15 mm | — | — | — |
| P/G + 25 wt % LG | — | 11 mm | 8 mm | — | — |
| P/G + 1 wt % ZnO + 25 wt % LG | 8 mm | 12 mm | 8 mm | 8 mm | 7 mm |

The data shown in the Table 2 suggest that emulsions containing essential oil and ZnO nanoparticles exhibit broader spectrum of antibacterial activity. This further indicates that there is a synergism of essential oils and metal oxide nanoparticles, such as ZnO nanoparticles, when considering antimicrobial properties of the emulsion according to the present invention.

### Mechanical Properties of Dried Solids Made of the Emulsion

Mechanical properties such as tensile strength, elongation-to-break, Young modulus of the material according to the present invention were also tested.

Table 3 shows the comparison of the mechanical properties of foils based on pectin/gelatin, in the presence of ZnO nanoparticles or Zn-acetate.

TABLE 3

| | | Tensile | Elongation- | Young |
|---|---|---|---|---|
| | | strength, | to-break, | modulus, |
| | SAMPLE | Rm (N/mm²) | A (%) | E (MPa) |
| 1. | P/G | 4.292 | 16.89 | 25.41 |
| 2. | P/G + ZnO | 18.816 | 16.01 | 117.52 |
| 3. | P/G + Zn-acetate | 39.508 | 25.32 | 156.32 |

From the data shown in the Table 3, it can be concluded that ZnO nanoparticles and Zn-acetate significantly improve elasticity of the foils and their mechanical properties in general.

### Slow Release of Essential Oils

UV-Vis spectrophotometer was used to determine LG oil content in emulsion during 8 days, by measuring the absorbance of citral as the major active component of LG oil, which is directly proportional to the concentration of LG oil in emulsion. The intensity values at 240 nm (which is the most intensive peak in absorption spectrum of LG oil) were used to determine the amount of LG oil in emulsion. UV-Vis spectra of pure LG oil were acquired with increasing amount of LG oil, and a calibration curve of the intensity of the peaks at 240 nm vs. the LG concentration was constructed. To determine amount of LG oil in emulsions, the intensity in UV-Vis spectrum at the 240 nm was extrapolated to the calibration curve.

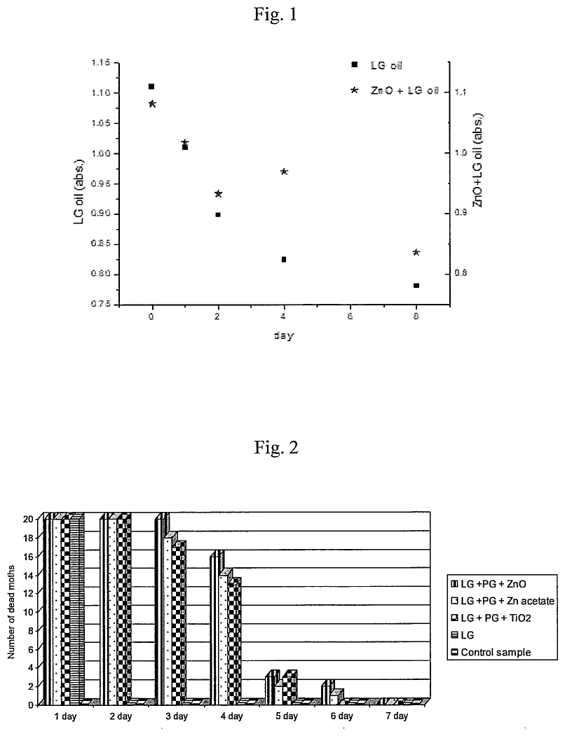

UV-VIS spectroscopy was used to measure the absorbtion of the active components of LG. Absorbtion was directly proportional to the concentration of LG (FIG. 1).

As shown in FIG. 1, the presence of ZnO nanoparticles reduced the release rate of the active component of LG during the period of 8 days.

FIG. 2. demonstrates the efficacy of the emulsions containing different nanoparticles or zinc acetate in comparison to pure LG essential oil tested against Ph. opercullela (potato tuber moth).

According to the results shown in FIG. 2, all tested samples (emulsions and pure LG essential oil) showed 100% efficacy against Ph. opercullela (potato tuber moth) in the first 24 h after application. On the other hand, pure essential oil was efficient only in the first 24 h while our emulsions showed prolonged effect up to 6 days after application. The best results regarding prolonged insecticide efficacy were obtained for emulsions containing ZnO nanoparticles. The above given results confirmed slow release of essential oils in our emulsions.

### Microstructure of Dried Thin Foils Made of the Emulsion

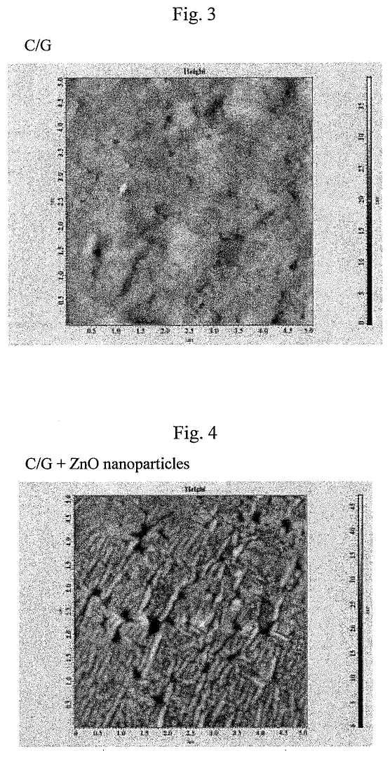

The microstructure of the foils was analyzed by atomic force microscope (AFM), as shown in FIG. 3. Biopolymer matrix consists of randomly distributed polymeric chains of chitosan and gelatin.

FIG. 4. shows that the presence of ZnO nanoparticles modifies the microstructure of the biopolymer matrix, forming clearly more organized structure.

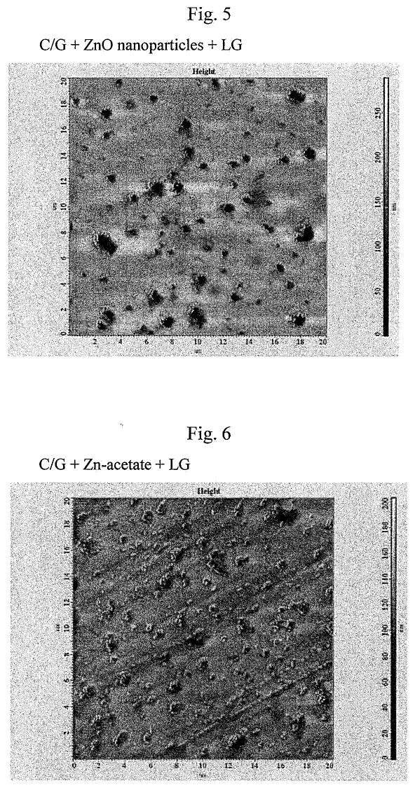

According to the AFM micrographs of the foils comprising C/G, ZnO nanoparticles and 25 wt % LG (FIG. 5.) and C/G, Zn-acetate and 25 wt % LG (FIG. 6.), LG droplets are uniformly distributed in biopolymer while ZnO nanoparticles/Zn-acetate is mostly arranged around nanoencapsulated droplets of LG, which confirms their role in slower rate of release of LG and its components.

1. Emulsion for active packaging containing:
biopolymer,
essential oil,
plasticizer,
surfactant, at least 15 wt % based on total weight of essential oil,
characterized by
further containing metal in the form of a salt, nanoparticles or metal oxide nanoparticles or combination thereof, and essential oil being encapsulated in biopolymer.

2. The emulsion according to claim 1 wherein the metal is selected from the group containing: silver, gold, zinc, titanium, calcium, copper and magnesium.

3. The emulsion according to claim 1 wherein the biopolymer is selected from the group comprising: polysaccharides, products, proteins, lipids and combination thereof.

4. The emulsion according to claim 1 wherein the plasticizer is selected from the group comprising glycerol, sorbitol, xylitol, PEG, PG, sucrose, fatty acids.

5. The emulsion according to claim 1 wherein the metal in the form of a salt is Zn-acetate.

6. The emulsion according to claim 1 wherein the metal oxide nanoparticles are selected from ZnO nanoparticles or TiO₂ nanoparticles.

7. The emulsion according to claim 1 wherein the essential oil is selected from the group comprising Allium sativum, Cinnamomum zeylanicum, Cuminum cyminum, Epilobium parviflorum, Lavandula officinalis, Mentha piperita, Ocimum basilicum, Ocimum gratissimum, Origanum majorana, Origanum vulgarae, Pimenta dioica, Pimpinella anisum, Piper betle, Psiadia arguta, Psiadia terebinthina, Rosmarinus officinalis, Salvia desoleana, Salvia sclarea, Satureja, Montana, Thymus vulgaris etc. or their active components: p-cymene, limonene, menthol, eugenol, anethole, estragole, geraniol, thymol, γ-terpinene, cinnamyl alcohol or combination thereof.

8. Spray made of the emulsion according to claim 1.

9. Pad made of the emulsion according to claim 1.

10. Foil made of the emulsion according to claim 1.

11. Coating for antibacterial protection of the product containing the emulsion according to claim 1.

12. Use of the emulsion according to claim 1 for product packaging.

13. Use of the emulsion according to claim 12 wherein the product is fresh fruit, vegetable, meat or cereal.

14. Use of the emulsion according to claim 1 as pesticide.

15. Method of preparing the emulsion according to claim 1, the method comprises the steps of
1) Dissolving the biopolymer in water
2) Adding a plasticizer to the biopolymer solution
3) Adding essential oil to the mixture of biopolymer solution and plasticizer
4) Homogenization
5) Adding at least 15 wt % surfactant based on total weight of the essential oil to the homogenized mixture obtained in step 4
6) Homogenization to form a stable emulsion
7) Adding the metal in the form of a salt, nanoparticles or metal oxide nanoparticles or combination thereof to the emulsion obtained in step 6
8) Homogenization
